(19) United States
(12) Patent Application Publication
(10) Pub. No.: US 2024/0115783 A1
(43) Pub. Date: Apr. 11, 2024

(54) AUGMENTATION OF GAS EXCHANGE BY AN ACOUSTICALLY OSCILLATING MEMBRANE

(71) Applicant: University of Pittsburgh - Of the Commonwealth System of Higher Education, Pittsburgh, PA (US)

(72) Inventors: Anthony L. Mercader, Pittsburgh, PA (US); Sung Kwon Cho, Pittsburgh, PA (US)

(21) Appl. No.: 18/480,928

(22) Filed: Oct. 4, 2023

Related U.S. Application Data
(60) Provisional application No. 63/413,367, filed on Oct. 5, 2022.

(51) Int. Cl. A61M 1/16; A61M 1/26

(52) U.S. Cl. CPC A61M 1/1698; A61M 1/1621; A61M 1/1678; A61M 1/26

(57) ABSTRACT

A device includes a housing, a gas inlet, a gas outlet, a liquid inlet, a liquid outlet, and one or more gas exchange units within the housing. Each gas exchange unit includes a gas channel in fluid connection with the gas inlet and with the gas outlet and a first liquid channel in fluid connection with the liquid inlet and with the liquid outlet. The first liquid channel is positioned adjacent to the gas channel and is separated from the gas channel via a first gas-permeable membrane. The first gas-permeable membrane is connected to a rigid substrate system so that the first gas-permeable membrane extends beyond a first edge of the rigid substrate system. The device further includes an oscillator to induce oscillation in the rigid substrate system and thereby in the first gas-permeable membrane.

### CROSS-REFERENCE TO RELATED APPLICATIONS

This application claims benefit of U.S. Provisional Patent Application Ser. No. 63/413,367, filed Oct. 5, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Recently, microfluidic systems have been proposed for a number of uses in many areas, including, for example, gas exchange in artificial lungs. In general, the term “microfluidics” refers to the behavior, precise control, and manipulation of fluids that are geometrically constrained to a very small scale at which surface forces dominate volumetric forces. In general, microchannels have a dimension or hydraulic diameter of less than a 1 mm. Typically, microchannels have a dimension in the range of 1-100 μm.

A recent trend in devices for gas exchange for lung assist is, for example, to mimic human lung structures using microchannels. The small dimensions of such microchannels result in a laminar flow and thus significant inhibition to mass transport. In the case of microfluidic systems used in artificial lungs in which gas exchange between a liquid (blood) and a carrier gas occurs through a gas-permeable membrane, microchannel heights are typically approximately 10-100 microns (10-100 μm). Under current designs, microchannel height is limited to ensure that diffusion reaches throughout the channel height.

Although microfluidic systems hold promise in, for example, artificial lungs, the limits on channel dimensions give rise to a number of significant problems. For example, plugging of channels is common. Moreover, high shear rates associated with small channels can lead to blood damage. Further, manufacturability is an issue at small scale. Further, to achieve suitable gas transfer rates in, for example, artificial lungs and other uses, thousands of microchannels may have to be stacked or otherwise combined in a gas exchange system, which is very difficult and significantly complicates manufacturing processes.

It is desirable to develop improved systems for gas exchange.

### SUMMARY

In one aspect, a device includes a housing, a gas inlet in connection with the housing, a gas outlet in connection with the housing, a liquid inlet in connection with the housing, a liquid outlet in connection with the housing, and one or more gas exchange units within the housing. Each of the gas exchange units includes a gas channel within the housing and in fluid connection with the gas inlet and with the gas outlet and either (i) a first liquid channel in fluid connection with the liquid inlet and with the liquid outlet or (ii) the first liquid channel and a second liquid channel in fluid connection with the liquid inlet and with the liquid outlet. The first liquid channel is positioned adjacent to the gas channel on a first side thereof. The second liquid channel, when present, is positioned adjacent to the gas channel on a second side thereof, opposite the first side. The first liquid channel is separated from the gas channel via a first gas-permeable membrane so that gas may transport between the first liquid channel and the gas channel via the first gas-permeable membrane. The second liquid channel, when present, is separated from the gas channel via a second gas-permeable membrane so that gas may transport between the second liquid channel and the gas channel via the second gas-permeable membrane. The first gas-permeable membrane is connected to a rigid substrate system so that the first gas-permeable membrane extends beyond a first edge of the rigid substrate system. The second gas-permeable membrane, when present, is connected to the rigid substrate system so that the second gas-permeable membrane extends beyond a second edge of the rigid substrate system. The device further includes an oscillator system comprising one or more oscillators in operative connection with the rigid substrate system. The oscillator system is configured to induce oscillation in the rigid substrate system and thereby in the first gas-permeable membrane and the second gas-permeable membrane, when present.

In a number of embodiments, each of the first liquid channel and the second liquid channel, when present, has a height no greater than 2 mm. Each of the first liquid channel and the second liquid channel, when present, may have a height of at least 50 mm or of a height of at least 200 mm. In a number of embodiments, each of the first liquid channel and the second liquid channel, when present, has a height in the range of 200 mm to 1 mm.

A frequency of oscillation of each of the one or more oscillators may, for example, be controlled to be in the range of 1 kHz to 20 kHz. A wavelength of oscillation induced in the first gas-permeable membrane may be greater than any dimension of the first liquid channel and greater than any dimension of the second liquid channel, when present. A wavelength of oscillation induced in the first gas-permeable membrane may, for example, be 10 times or 100 times greater than any dimension of the first liquid channel and greater than any dimension of the second liquid channel, when present. In a number of embodiments, a wavelength of oscillation is at least 10 times the dimension of the first liquid channel in the (primary) direction in which waves oscillate through the first gas-permeable membrane, and at least 10 times the dimension of the second liquid channel, when present, in the (primary) direction in which waves oscillate through the second gas permeable membrane. In a number of embodiments, a wavelength of oscillation is at least 100 times the dimension of the first liquid channel in the (primary) direction in which waves oscillate through the first gas-permeable membrane, and at least 100 times the dimension of the second liquid channel, when present, in the (primary) direction in which waves oscillate through the second gas permeable membrane.

In a number of embodiments, a direction of bulk flow of gas through the gas channel is oriented generally perpendicular to bulk flow of liquid through the first liquid channel and through the second liquid channel, when present.

In a number of embodiments, the first gas-permeable membrane extends from a first edge of a first section of the rigid substrate system, and the second gas-permeable membrane extends from a second edge of the first section of the rigid substrate system, which is opposite the first edge. A space between a first section of the rigid substrate system and a second section of the rigid substrate system may form the gas channel. The first gas-permeable membrane may, for example, be connected to the first section and to the second section of the rigid substrate system to span the gas channel.

In a number of embodiments, the device includes a plurality of the gas exchange units. The plurality of gas exchange units may be positioned in a stacked arrangement. In a number of embodiments, each of the plurality of gas exchange units has a separate oscillator in operative connection with the rigid substrate system thereof. In a number of embodiments, an oscillator is in operative connection with rigid substrate of more than one of the plurality of gas exchange units.

In another aspect, a method of effecting gas exchange between a liquid and a sweep gas includes providing a device hereof. The device, for example, includes a housing, a gas inlet in connection with the housing, a gas outlet in connection with the housing, a liquid inlet in connection with the housing, a liquid outlet in connection with the housing, and one or more gas exchange units within the housing. Each of the gas exchange units includes a gas channel within the housing and in fluid connection with the gas inlet and with the gas outlet and either (i) a first liquid channel in fluid connection with the liquid inlet and with the liquid outlet or (ii) the first liquid channel and a second liquid channel in fluid connection with the liquid inlet and with the liquid outlet. The first liquid channel is positioned adjacent to the gas channel on a first side thereof. The second liquid channel, when present, is positioned adjacent to the gas channel on a second side thereof, opposite the first side. The first liquid channel is separated from the gas channel via a first gas-permeable membrane so that gas may transport between the first liquid channel and the gas channel via the first gas-permeable membrane. The second liquid channel, when present, is separated from the gas channel via a second gas-permeable membrane so that gas may transport between the second liquid channel and the gas channel via the second gas-permeable membrane. The first gas-permeable membrane is connected to a rigid substrate system so that the first gas-permeable membrane extends beyond a first edge of the rigid substrate system. The second gas-permeable membrane, when present, is connected to the rigid substrate system so that the second gas-permeable membrane extends beyond a second edge of the rigid substrate system. The device further includes an oscillator system comprising one or more oscillators in operative connection with the rigid substrate system. The oscillator system is configured to induce oscillation in the rigid substrate system and thereby in the first gas-permeable membrane and the second gas-permeable membrane, when present. The method further includes passing liquid through the first liquid channel and the second liquid channel, when present, via the liquid inlet and the liquid outlet; and passing gas through the gas channel via the gas inlet and the gas outlet. The liquid may, for example, include blood and the gas include oxygen.

In a number of embodiments, each of the first liquid channel and the second liquid channel, when present, has a height no greater than 2 mm. Each of the first liquid channel and the second liquid channel, when present, may have a height of at least 50 mm or of a height of at least 200 mm. In a number of embodiments, each of the first liquid channel and the second liquid channel, when present, has a height in the range of 200 mm to 1 mm.

A frequency of oscillation of each of the one or more oscillators may, for example, be controlled to be in the range of 1 kHz to 20 kHz. A wavelength of oscillation induced in the first gas-permeable membrane may be greater than any dimension of the first liquid channel and greater than any dimension of the second liquid channel, when present. A wavelength of oscillation induced in the first Gas-permeable membrane may, for example, be 10 times or 100 times greater than any dimension of the first liquid channel and greater than any dimension of the second liquid channel, when present. In a number of embodiments, a wavelength of oscillation is at least 10 times the dimension of the first liquid channel in the (primary) direction in which waves oscillate through the first gas-permeable membrane, and at least 10 times the dimension of the second liquid channel, when present, in the (primary) direction in which waves oscillate through the second gas permeable membrane. In a number of embodiments, a wavelength of oscillation is at least 100 times the dimension of the first liquid channel in the (primary) direction in which waves oscillate through the first gas-permeable membrane, and at least 100 times the dimension of the second liquid channel, when present, in the (primary) direction in which waves oscillate through the second gas permeable membrane.

In a number of embodiments, a direction of bulk flow of gas through the gas channel is oriented generally perpendicular to bulk flow of liquid through the first liquid channel and through the second liquid channel, when present.

In a number of embodiments, the first gas-permeable membrane extends from a first edge of a first section of the rigid substrate system, and the second gas-permeable membrane extends from a second edge of the first section of the rigid substrate system, which is opposite the first edge. A space between a first section of the rigid substrate system and a second section of the rigid substrate system may form the gas channel. The first gas-permeable membrane may, for example, be connected to the first section and to the second section of the rigid substrate system to span the gas channel.

In a number of embodiments, the device includes a plurality of the gas exchange units. The plurality of gas exchange units may be positioned in a stacked arrangement. In a number of embodiments, each of the plurality of gas exchange units has a separate oscillator in operative connection with the rigid substrate system thereof. In a number of embodiments, an oscillator is in operative connection with rigid substrate of more than one of the plurality of gas exchange units.

In a further aspect, a system includes a plurality of devices, each of the plurality of devices includes a housing, a gas inlet in connection with the housing, a gas outlet in connection with the housing, a liquid inlet in connection with the housing, a liquid outlet in connection with the housing, and one or more gas exchange units within the housing. Each of the gas exchange units includes a gas channel within the housing and in fluid connection with the gas inlet and with the gas outlet and either (i) a first liquid channel in fluid connection with the liquid inlet and with the liquid outlet or (ii) the first liquid channel and a second liquid channel in fluid connection with the liquid inlet and with the liquid outlet. The first liquid channel is positioned adjacent to the gas channel on a first side thereof. The second liquid channel, when present, is positioned adjacent to the gas channel on a second side thereof, opposite the first side. The first liquid channel is separated from the gas channel via a first gas-permeable membrane so that gas may transport between the first liquid channel and the gas channel via the first gas-permeable membrane. The second liquid channel, when present, is separated from the gas channel via a second gas-permeable membrane so that gas may transport between the second liquid channel and the gas channel via the second gas-permeable membrane. The first gas-permeable membrane is connected to a rigid substrate system so that the first gas-permeable membrane extends beyond a first edge of the rigid substrate system. The second gas-permeable membrane, when present, is connected to the rigid substrate system so that the second gas-permeable membrane extends beyond a second edge of the rigid substrate system. The device further includes an oscillator system comprising one or more oscillators in operative connection with the rigid substrate system. The oscillator system is configured to induce oscillation in the rigid substrate system and thereby in the first gas-permeable membrane and the second gas-permeable membrane, when present.

In still a further aspect, a method of providing lung assist include passing blood of a patient through liquid channels of a device or a system hereof and passing a sweep gas including oxygen through the gas channels of the device or the system hereof.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a schematic diagram of an embodiment of a device, illustrated in cross section, hereof to achieve augmented or enhanced gas exchange via microstreaming flow created by an oscillating membrane wherein the bulk flow of liquid through the liquid channel is parallel to the direction of the oscillating wave in the membrane.

FIG. 1B illustrates an embodiment of a fabrication or manufacturing process for the device of FIG. 1A wherein the various stages of fabrication are illustrated in cross section.

FIG. 1C illustrates a perspective view of an experimental setup for an embodiment of a studied device hereof, wherein the bulk flow of liquid through the liquid channel is perpendicular to the direction of the oscillating wave in the membrane.

FIG. 1D illustrates a cross-sectional view an embodiment of a device hereof wherein a microstreaming flow channel extends from or hangs over an edge of a substrate such that the gas-permeable membrane thereof contacts the gas flow.

FIG. 1E illustrates schematically an embodiment of an experimental setup for an embodiment of a studied device similar to that of FIG. 1A wherein a tabletop vibration shaker is used as an oscillator/actuator instead of a piezo actuator/buzzer.

FIG. 2A illustrates a graph of experimentally determined magnitude of membrane oscillation as determined by Laser Doppler Vibrometer (LDV).

FIG. 2B illustrates a graph of experimentally determined phase of membrane oscillation as determined by LDV.

FIG. 3A illustrates a graph of acoustic streaming velocity and streamlines measured by Particle Image Velocimetry (PIV) for a 3 mm gap.

FIG. 3B illustrates a graph of acoustic streaming velocity and streamlines measured by PIV for a 1.5 mm gap.

FIG. 3C illustrates computational fluid dynamics (CFD) results given membrane oscillation input for a 3 mm gap for comparison with FIG. 3A.

FIG. 3D illustrates a computational fluid dynamics (CFD) results given membrane oscillation input for a 1.5 mm gap for comparison with FIG. 3B.

FIG. 4A illustrates an enlarged view of the results of experimental measurement of velocity field and streamline for the 1.5 mm membrane length with no infusion of streamwise liquid flow (as illustrated in FIG. 3C) with the primary and secondary vortices marked.

FIG. 4B illustrates an enlarged view of CFD results given membrane oscillation as input with no infusion of streamwise liquid flow (as illustrated in FIG. 3D) with the primary and secondary vortices marked.

FIG. 5A illustrates measured pH level (H⁺ concentration) in the liquid channel of a device hereof as an indicator of how much the CO₂ gas is transferred through the membrane to the liquid channel at a flow rate of 0.2 mL/min for a 1 mm channel height and a 250 μm channel height.

FIG. 5B illustrates measured pH level (H⁺ concentration) in the liquid channel of a device hereof as an indicator of how much the CO₂ gas is transferred through the membrane to the liquid channel at a flow rate of 0.4 mL/min for a 1 mm channel height and a 250 μm channel height.

FIG. 6A illustrates schematically and in cross section a representative embodiment of a gas exchange device hereof comprising multiple, stacked channels for gas exchange via a gas-permeable membrane.

FIG. 6B illustrates an exploded perspective view of another representative embodiment of a gas exchange device hereof comprising multiple, stacked channels for gas exchange via a gas-permeable membrane.

FIG. 7A illustrates the results of a study of blockage resulting from blood flow in actuated versus unactuated devices hereof.

FIG. 7B illustrates the results of a study of blockage resulting from blood flow in two actuated devices hereof with different microchannel heights.

FIG. 8 illustrates the results of a study of inlet pressure over time resulting from blood flow through two devices hereof with different microchannel heights.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a channel” includes a plurality of such channels and equivalents thereof known to those skilled in the art, and so forth, and reference to “the channel” is a reference to one or more such channels and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronics”, “electronic circuitry”, “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

In a number of embodiments hereof, a device configuration is provided which generates significant or relatively strong acoustic streaming in a microchannel/channel at, for example, an audible frequency range. In a number of studies hereof, the flow field in such a device was characterized and the underlying mechanisms thereof analyzed via advanced measurement techniques.

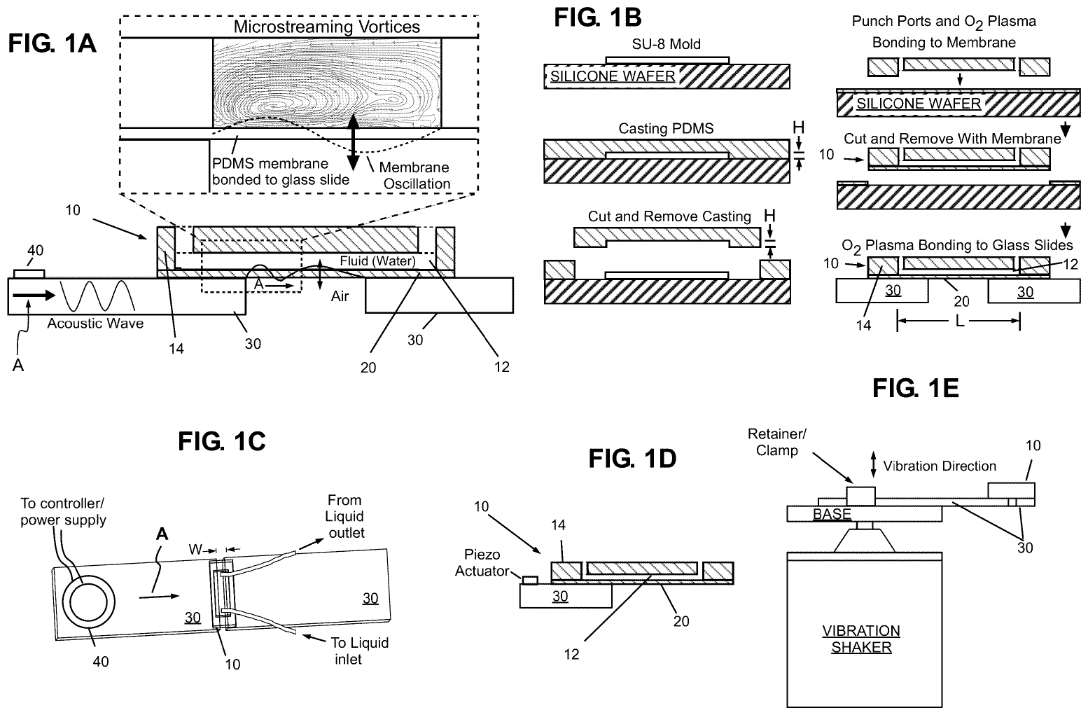

In a number of studied embodiments of a device hereof as illustrated, for example, in FIGS. 1A through 1C, a liquid channel or microchannel 12 is formed in a microchannel body 14 of a microchannel assembly 10. Microchannel body 12 is formed from, for example, a polymeric material (for example, a polydimethylsiloxane (PDMS), a polycarbonate, a polypropylene, etc.). A gas porous or permeable membrane 20 (for example, a PDMS membrane) is included as a bottom (in the illustrated orientation) surface or wall in forming microchannel assembly 10 (see, for example, FIGS. 1A, 1B and ID). In general, gases may readily pass through gas-permeable membranes 20 hereof while the transfer of liquid therethrough is significantly limited or prevented. Examples of suitable gas-permeable membrane materials other than PDMS include microporous polycarbonate (PC) and microporous polypropylene (PP). As described above, the material for microchannel body 12 is typically a polymeric material. Polymeric materials may, for example, facilitates manufacture. Any material suitable to form a microchannel therein through which fluid can flow may, however, be used in forming the microchannel body. Biocompatibility is desirable for use in connection with, for example, gas exchange in biological fluids such as blood. Hemocompatibility is further desirable for use in connection with blood. Chemical inertness with respect to the liquid is also desirable. PDMS is commonly used in many applications as it provides for ready manufacture via, for example, casting/curing and also provides for very good chemical inertness, biocompatibility/non-toxicity, and hemocompatibility.

In a number of studies, a portion of the bottom surface of membrane 20 was bonded/sealed to a substrate or substrate section 30. The substrate may, for example, be a material which is suitable to transmit vibration or an acoustic wave therethrough to membrane 20. Such materials may, for example, have low acoustic impedance. In general, relatively stiff materials with low acoustic impedance are suitable for use as a substrate herein. In a number of embodiments, the substrate has an elastic modulus of 30 GPa. Glass may, for example, have an elastic modulus of approximately 60 GPa. As substrate 30 does not contact the working fluid/liquid, chemical, biocompatibility, and hemocompatibility properties are less important. In the embodiments illustrated in FIGS. 1A through 1C, two spaced substrates or substrate sections 30 of rigid material (glass in the illustrated embodiment) are provided and channel 12/gas-permeable membrane 20 of microchannel assembly 10 extends past or hangs off the edge of each of spaced substrate sections 30 (with microchannel 12/gas-permeable membrane 20 spanning the gas channel formed therebetween), exposing membrane 20 to open air on the gas/air side of membrane 20 (see FIG. 1A). In the embodiment of FIG. 1D, a single substrate section 30 is provide and membrane 20 of microchannel assembly 10 as described in connection with FIG. 1B hangs over the edge thereof.

As describe above, the rigid/stiff substrate material is suitable to transmit vibration/acoustic waves. When an acoustic wave is transmitted through one or more of the rigid sections or substrates 30 operatively attached to membrane 20, it is focused on the edge thereof where membrane 20 is attached/pinned thereto, creating strong membrane oscillations and thus strong vortex flow patterns in liquid channel 12 above membrane 20 (see enlarged section of studied device showing microstreaming vortices in FIG. 1A). Using such a mechanism, a gas transfer device was designed where the permeable membrane separated liquid and gas channels, and the streaming flow in the liquid channel (induced by an actuator/oscillator) enhanced mixing in the liquid flow. Such microstreaming provided increased mass (gas) transfer which would normally be limited to diffusion on the liquid side of the membrane. The oscillatory/acoustic input creates a mechanical oscillation in the membrane since the wavelength of the acoustic input is much longer than the characteristic dimensions of the device.

The dimensions (height H, length, L, and width W; wherein W is dimension of channel 12 perpendicular to H and L in, for example, FIG. 1B) of liquid channel 12 may be considered to be the characteristic dimensions of the device hereof. In general, of those dimensions, the dimension of liquid channel 12 in the direction in which the acoustic wave travels may be considered the most characteristic dimension for defining the nature of the oscillations induced in gas-permeable membrane 20. The direction in which the acoustic waves travels is indicated by arrow A in FIGS. 1A and 1C. Thus, the most characteristic direction is L in the studied embodiment of FIG. 1A and W in the studied embodiment of FIG. 1C. Typically, however, each of the dimensions are of approximately the same order of magnitude in devices hereof. In a number of embodiments hereof, the wavelength of the acoustic input passing through gas-permeable membrane 20 is at least an order of magnitude (that is, 10×) greater than that most characteristic dimension (that is, is the dimension of liquid channel 12 in the direction the acoustic wave travels through membrane 20). In a number of embodiments, the wavelength is at least two orders of magnitude (that is, 100×) greater than that most characteristic dimension.

The devices, systems, and methods hereof do not create obstructions in the fluid field. In that regard, microstreaming by sharp-edge oscillation is generally of lower strength and necessitates an obstruction in the fluid field. See, for example, P.-H. Huang et al., Lab Chip., 13 no. 19 (2013) pp. 3874-3852. Furthermore, there is no issue with bubble dissolution. In general, bubbles may be, and typically are, absent from the liquid channel(s) hereof. In that regard, the configuration of devices hereof does not suffer from stability issues related to dissolution of the driving mechanism that occur with bubble microstreaming. While microstreaming by acoustically oscillating bubbles is quite strong, it may suffer from the stability issue arising from bubble dissolution. See, for example, P. Tho et al., J. Fluid Mech., 573 (2007) pp. 191-233. Furthermore, blood is not compatible with direct bubble contact.

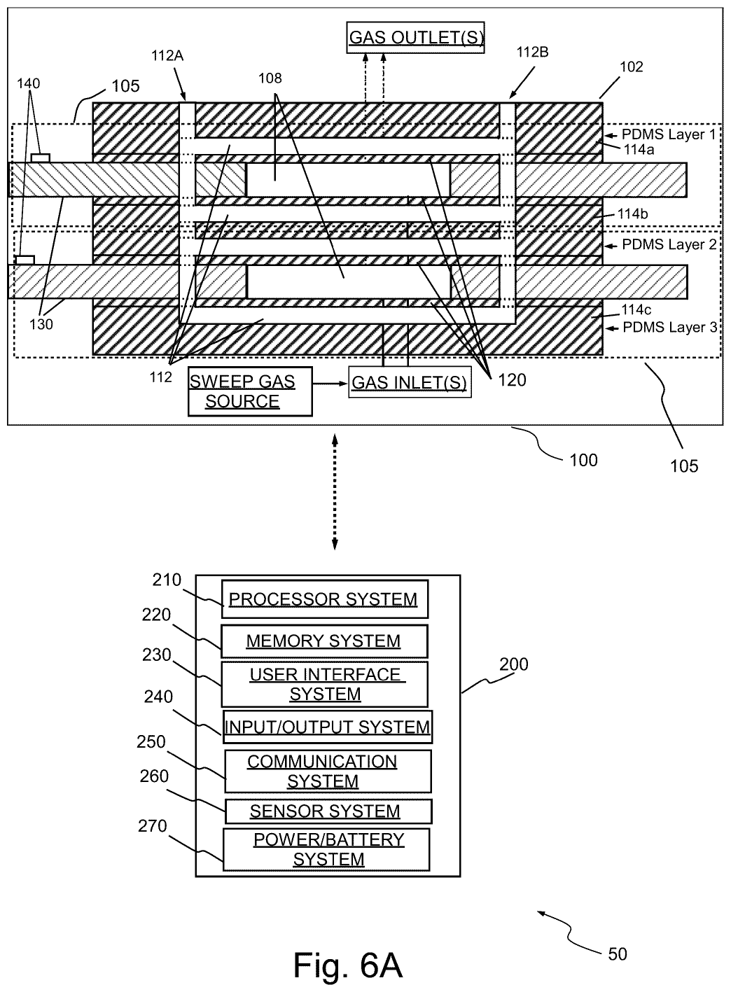

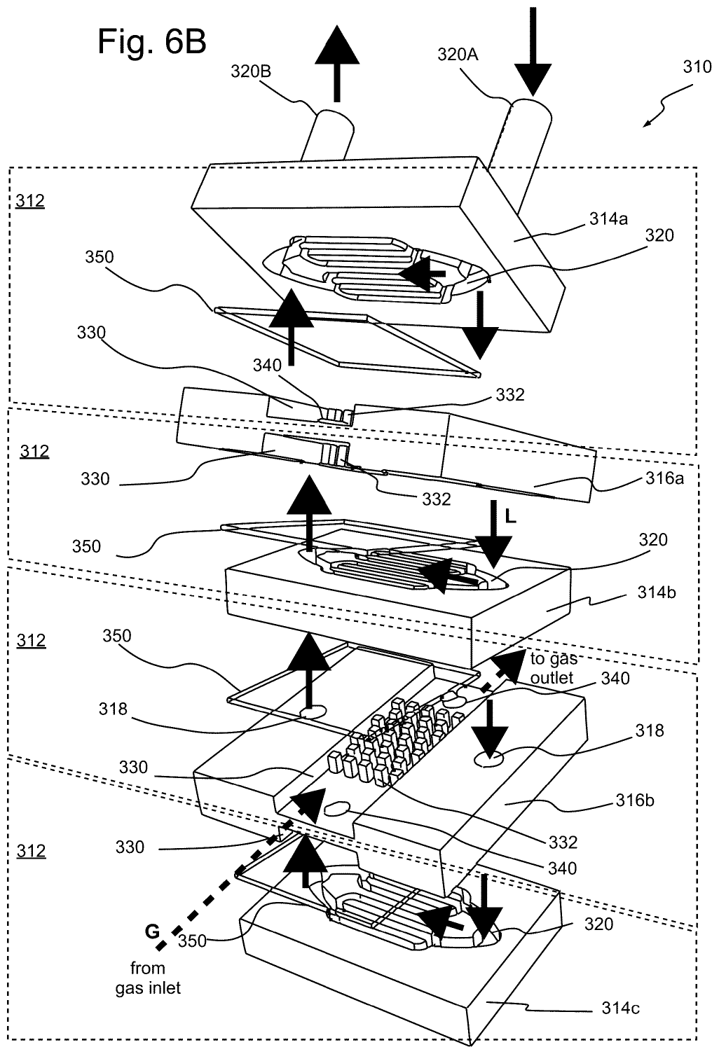

A representative example of a fabrication process for fabricating microchannel assemblies 10 hereof is illustrated in FIG. 1B. In that regard, a standard soft lithography process was used in the fabrication of the representative devices of FIGS. 1A through IB (which include a “pinned” gas-permeable membrane 20 to generate acoustic streaming as described above). First, a spin coater was used to spread SU-8 2075 (an epoxy-based, negative photoresist) in an even layer on top of a silicon wafer up to a maximum of 300 μm depending on spin speed. The resin was cured on a hot plate and the process was repeated in multiple layers for heights channel heights H (that is, the distance between the membrane and the distal/top end of the liquid channel; see FIG. 1B) above 300 μm. The photoresist was exposed to UV light in a mask aligner in an area defined by a photolithography mask in the shape of the top view of the liquid channel (see, for example, FIG. 6B for a representative liquid channel design). The exposed photoresist was submerged in SU-8 developer chemical to remove the area of SU-8 which was not exposed to UV light, leaving a negative mold of the liquid channel. PDMS elastomer base and curing agent was mixed in a standard 10:1 ratio, poured over the mold, and cured at 90° C. for 30 minutes. The casting was then cut and removed from the mold by hand and the inlet and outlet hole were created by a 1 mm biopsy punch.

The studied membranes were fabricated by first coating an Si wafer with a 1 μm layer of Parylene-C (a polymer having a backbone of para-benzenediyl rings —C₆H₄— connected by 1,2-ethanediyl bridges —CH₂—CH₂—, and wherein one hydrogen atom in the aryl ring replaced by chlorine) by Chemical Vapor Deposition (CVD) to reduce adhesion. PDMS was then deposited on top in a 20 μm layer by spin coating and cured as described above. The bottom face/surface of the channel cutout and the membrane were then chemically bonded. In that regard, a handheld air plasma generator was held above the surfaces to be bonded together for 1 minute each. Subsequently, the channel cutout was pressed onto the membrane surface and left overnight. Air or O₂ plasma may be used. The membrane with bonded channel cutout was then cut away from the Si/Parylene substrate with a razor blade. The bonding process was then repeated as described above to bond the underside of the membrane between two glass sections, substrates, or slides with a desired gap therebetween. In a number of embodiments, a suitable thickness range for the gas-permeable membrane is approximately 1-100 μm.

In a number of studies, a piezo oscillator or actuator 40 (for example, a piezo buzzer) was attached to one of the glass slides/substrate sections 30 by epoxy to transmit the acoustic waves given, for example, a sinusoidal voltage input from a function generator and amplifier. Any oscillator or actuator configured to create oscillation or vibration, as known in the art, can be used herein. As used herein, an “actuator” is a device that converts electrical energy into mechanical force/movement. To adapt the described design for gas transfer experiments, another section of PDMS was bonded to the underside of the sections of rigid substrates (glass slides) to seal another channel to be used for gas flow in a number of studies. As, for example, illustrated in FIG. 1D, which is described further below, a vibration shaker may be used as an actuator herein.

The devices hereof, which may, for example, provide acoustic streaming flow, are relatively inexpensive to fabricate and operate, meaning, for example, that they can be readily integrated into certain existing microchannel-based devices. Because of the necessity of an exposed membrane and vertical orientation of the mixing, gas transfer in a microfluidic artificial lung device is a representative example of an application of the devices, systems and methods hereof See, for example, J. A. Potkay, Lab Chip., 14 no. 21 (2014) pp. 4122-4138. The vortices created by the oscillating, gas-permeable membranes hereof may be used to enhance mass transfer, which would normally be limited by diffusion within the channel/microchannel.

As described above, advanced measurement techniques were used to characterize the novel flow patterns created in the devices, systems, and methods hereof to develop a detailed description of the flow field and to investigate the driving mechanisms thereof. A system of co- or counterrotating vortices was found to span the area of membrane oscillation which took shape as a superposition of standing and travelling waves. Such boundary oscillations were applied to a CFD simulation, and the resulting time-averaged velocity field showed good agreement with the micro Particle Image Velocimetry (PIV) measured velocity field, providing evidence that the membrane oscillation is the primary mechanism driving the streaming vortices.

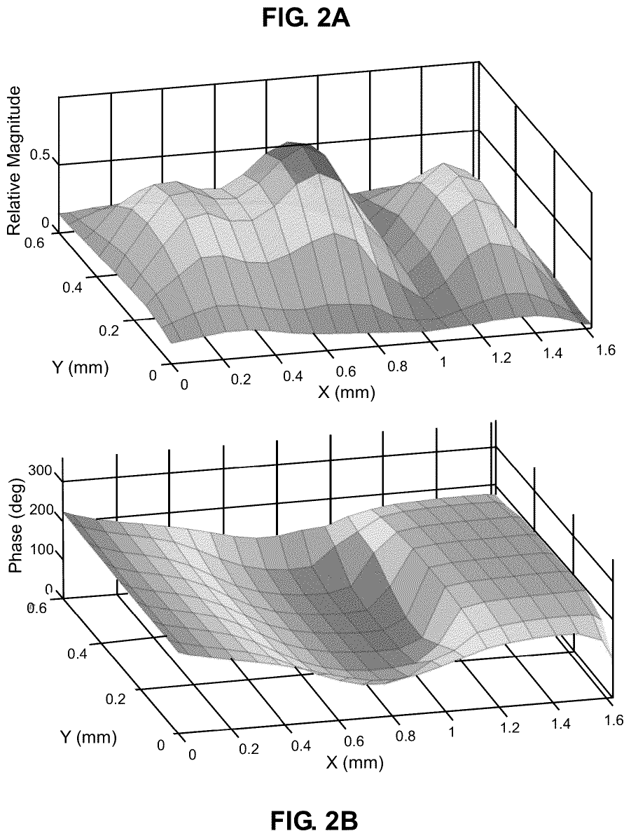

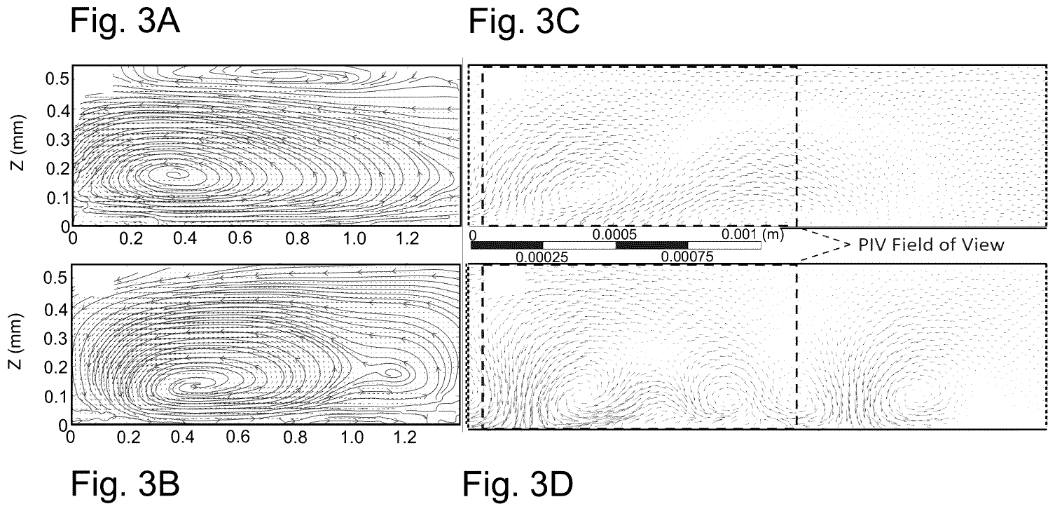

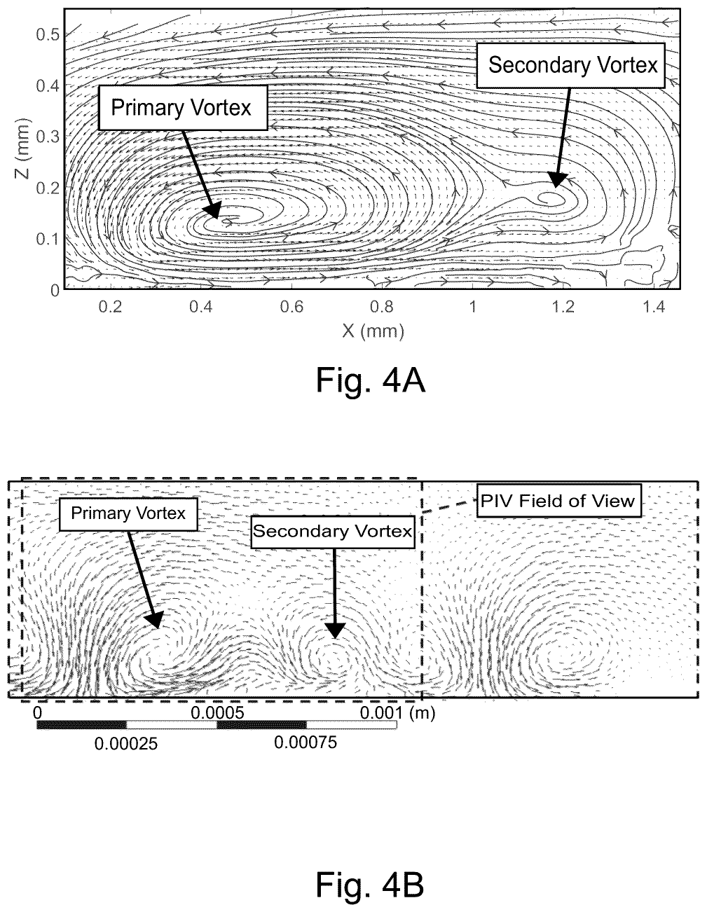

Laser Doppler Vibrometry (LDV) was, for example, used to characterize the membrane oscillation. As described above, the observed pattern was a superposition of travelling and standing waves depending on the length of membrane exposed to open air (FIGS. 2A and 2B, respectively) with approximately linear scaling with input voltage. A peak displacement is 18.5 μm for 20 Vₚₚ. PIV was used to measure the velocity field and vortices (FIGS. 3A, 3B and 4A) without any main streamwise flow in the microchannel. The induced microstreaming velocity in the microchannel quadratically scaled with input voltage reaching up to 47 mm/s for 20 Vₚₚ. The LDV data were applied as a boundary condition for CFD simulation with the ANSYS® CFX® solver (engineering simulation software, available from ANSYS, Inc. of San Jose, California). The time-average flow field showed good agreement with the Ply results in vortex pattern and magnitude (FIG. 4B) providing evidence that the mechanical oscillation of the membrane, not the acoustic field itself, is the driving mechanism of the microstreaming vortices. Next, the main streamwise flow was infused into the micro channel by a syringe pump so the axes of rotation of the vortices were aligned with the streamwise flow, creating helical streamlines in the channel. Further description of the experimental characterizations of the device hereof is provided below.

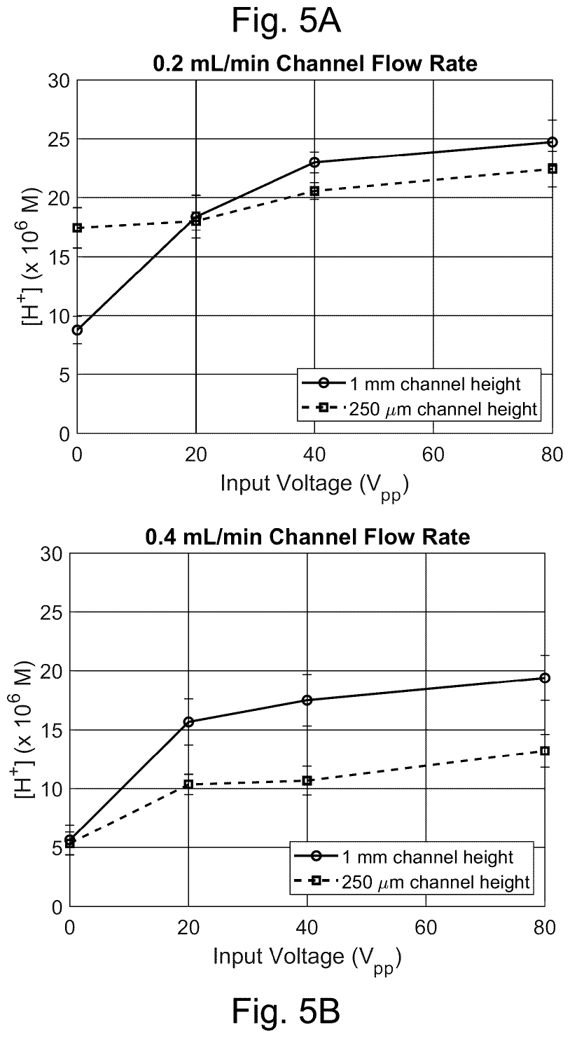

To experimentally measure gas transfer, a CO₂ gas stream was interfaced below the PDMS membrane. The measured pH level (H⁺ concentration) in the microchannel was an indicator of how much the CO₂ gas was transferred through the membrane to the microchannel/channel (see FIGS. 5A and 5B). Comparing channels of 250 μm and 1 mm heights, at no actuation, CO₂ transfer through the permeable membrane is greater or equal for the shorter channel at any flow rate. At any level of actuation, the taller channel outperforms the shorter channel, up to 3.4× compared to no actuation, indicating that the design of devices hereof provides the dual benefit of: (1) improved gas transfer and (2) larger channel height to enhance hemocompatibility as a result of significantly reduced shear and to, for example, facilitate scale-up fabrication towards, for example, microfluidic artificial lung technology.

In the LDV studies hereof, the LDV measurement was performed on the section of the membrane exposed to open air to characterize its oscillation pattern in magnitude and phase. The microchannel was filled with water and sealed shut. The underside of the membrane was aligned with the view of the microscope objective and a grid of measurement points was specified over the area of interest. Once again, the observed pattern was a superposition of travelling and standing waves starting at the glass edge from which the acoustic waves originate. The travelling wave component dominates near that same edge and gives way to a standing wave dominant portion close to the opposite glass edge, evidenced by the steady decrease in phase starting from the leading edge and a flattening out of the plot on the opposite side. The largest velocity gradient and magnitude appeared nearest the primary glass edge, which corresponds to the main vortex and fastest velocity observed in the PIV experiments. As described above, the displacement magnitude varied approximately linearly with voltage, and the peak displacement observed was 18.5 μm for a 20 Vpp input. The wavelength observed in the membrane oscillation of close to 1.5 mm was much shorter than the acoustic wavelength in PDMS, which would be 196 mm at the actuation frequency of 5.5 kHz wherein wavelength in m, vis wave velocity in m/s (through the material in which wavelength is being measured), and f is frequency in Hz−), indicating that the pattern is mechanical in nature, not acoustic. This observation can be seen as a reason the acoustic streaming occurs at a lower frequency in the studies hereof as compared to ultrasound configurations. In general, the input frequencies into the oscillator hereof are such that the resultant wavelength is greater (and typically significantly greater) than any dimension of the devices hereof as described above. Input frequencies hereof may, for example, be in the acoustic range of 1 kHz to 20 kHz.

For the PIV studies, a 600 μm×600 μm microchannel fabricated as described above was seeded with fluorescent particles and a sine wave acoustic signal was transmitted therethrough. The particles were illuminated by laser light reflected by a right-angle prism to generate a side view. For two separate pulses with known time delay, two images corresponding to those laser pulses were correlated by PIA/software, generating a velocity vector field in that field of view.

The PIV data shown in FIGS. 3A, 3B and 4A give a clear picture of the streaming patterns for the entire channel height and within approximately 1.5 mm down the channel length (FIGS. 3B and 4A) based on the field of view of the system. In the case of 3 mm length (FIG. 3A) of membrane determined by the gap between glass slides, a single strong vortex was captured in the field of view which spanned the height of the channel with the highest velocity located near the membrane. For the 1.5 mm long membrane case (FIG. 3B), a view of the entire flow field was possible which revealed a three-vortex system. There was a similar main vortex spanning the height of the channel, a much slower corotating vortex within the main one, and a third counterrotating vortex bound by the opposite glass. Noting the maximum velocity at the bottom of the main vortex, the scaling with input voltage was approximately quadratic. As described above, at 20 Vpp, a maximum velocity of 47 mm/s was measured.

CFD simulations (see FIGS. 3C, 3D and 4B) were executed with the ANSYS CFX solver to further investigate the driving mechanism of the streaming flow. Geometry was created with dimensions to match the fabricated channels. Boundary conditions were applied to that geometry where the inlet and outlet were left open, the top and side walls were set to no-slip, and the bottom wall representing the membrane was set as a no-slip wall with specified displacement in time corresponding to the magnitude and phase data from LDV. The streaming flow was visualized by calculating the time average of the velocity field over the time steps in the final acoustic period. The simulation results are shown in FIGS. 3C, 3D and 4B show good agreement in pattern and magnitude to the PIV results, providing evidence that the mechanical oscillation of the membrane was the driving mechanism of the acoustic streaming vortices. The three-vortex system was correctly predicted by simulation for the 1.5 mm gap case (FIGS. 3D and 4B). The input amplitude of displacement for the simulation was related to the input voltage in the PIV experiment through the LDV results and shows similar quadratic scaling. The velocity was predicted well, though slightly higher than experiment by close to 15% at higher input amplitudes, potentially as a result of sample-to-sample variation.

The devices, systems, and methods hereof generate acoustic microstreaming at audible frequency. Through advanced measurement techniques and simulation, flow in devices hereof was characterized as described above, providing evidence that the driving mechanism was the oscillating membrane. The devices, systems, and methods studied herein where not optimized, and further optimization may be achieved. The interaction of the patterns with streamwise flow and the effect of orientation of the oscillation may, for example, be characterized. As set forth above, the devices, systems, and methods hereof may be applied to gas transfer towards, for example, a micro artificial lung device.

As described above, in addition to standard piezoelectric actuators described in connection with a number of studies hereof, a tabletop vibration shaker (Ling Dynamic Systems V200 series available from Brüel and Kjaer of Naerum, Denmark) was investigated as an alternative method of actuation to generate streaming in a microchannel by an oscillating membrane (see FIG. 1D). Such an actuator may sacrifice compactness in favor of flexibility. A piezoelectric actuator or buzzer has a nominal resonant frequency depending on the specific model. Moreover, the substrate will have certain resonant frequencies depending on the geometry. These factors are not independently tunable once the device is fabricated and the piezoelectric actuator is attached to the substrate, potentially leading to reduced performance if the frequencies are not well matched. In the case of a vibration shaker, the response is more even over the range of frequencies being investigated and less dependent on resonance. Also, the devices are attached to the shaker via a custom sample holder which allows for tuning of the resonant frequency of the substrate quickly by changing the positioning of the substrate on the holder. As illustrated in FIG. 1E, one of the glass slides, substrates, or sections as described in connection with FIG. 1C, was large enough to be clamped (via two clamps in the illustrated embodiment) to a base which was attached to a vibration shaker. Further, a sample holder may be readily designed to hold more than one rigid substrate (for example, in a stacked configuration similar to that illustrated in FIGS. 6A and 6B), meaning that multiple devices could be actuated at once using only one vibration shaker. To the contrary, in the “vertical stack” concept described in connection with FIGS. 6A and 6B, each layer may require its own piezoelectric actuator.

Studies hereof have demonstrated that streaming vortices can be generated in a microchannel using a vibration shaker such as the LDS V200. Streaming has been observed with this configuration over a studied range of approximately 600-2000 Hz. Material and fabrication methodologies may be optimized further to strengthen the streaming effect for enhanced gas transfer.

The fact that the acoustic streaming occurs at or around audible frequency distinguishes the devices, systems and methods hereof from other devices, systems, and methods because, in this range, the acoustic wavelength is much larger than any characteristic dimension in the system. In representative studies of the devices hereof, the frequency range was between 5 and 7.5 kHz (acoustic wavelength of 30 and 20 cm in water, respectively). For the perpendicular orientation used for gas transfer studies (see FIG. 1C), the channel length (in a direction parallel to the axis of rotation of the vortices) was 1.5 cm, the channel width (parallel to the acoustic wave travelling direction) was 1.6 mm, and the channel height ranged between 200 and 1 mm.

As known in the artificial lung or lung assist arts, many design considerations including, for example, pressure drop, blood stability, gas exchange requirements, saturation rate of gas transfer through the membrane, etc., must be considered in designing lung assist devices including channels as described herein. In general, the height of liquid channels herein for use in such a device may, for example, be in the range of approximately 10 μm to 2 mm, 50 μm to 2 mm, 50 μm to 1 mm, 100 μm to 1 mm or, 200 μm to 1 mm. As channel height increases, it may be desirable to increase oscillation amplitude. Channel cross-sectional area, as well as channel length affect the required pressure drop across a channel. If no powered pump is present in a system, the pressure is limited to approximately 80 mmHg, which is the pressure supplied by the human heart. Power pump system may be used to provide additional pressure. One skilled in the art can readily design a lung assist system using channel designs as described herein to achieve a desire gas exchange rate. In addition to the ranges of channel height as describe above, the length of the channel (referring to an embodiment in which the acoustic wave travels perpendicular to that length as, for example, illustrated in FIG. 1C) may be in the range of approximately 1 cm to approximately 10 cm in a number of embodiments. The width may, for example, be in the range of approximately 1 mm to approximately 5 mm or approximately 1 mm to approximately 3 mm.

The above principle is applicable for a wider range of frequencies where the acoustic wavelength is quite long if the resulting membrane oscillation amplitude is large enough. The relationship may be weaker at lower frequencies as a result of the lower vibration velocity for a given amplitude, leading to a practical lower limit. The frequencies described above were chosen for the studies hereof because they were close to the mechanical resonant frequencies of both the piezo oscillator/actuator and the membrane itself, and therefore led to the highest membrane amplitude and acoustic streaming velocity. Suitable frequencies for various devices hereof are readily determined by those skilled in the art using engineering principles and routine experimentation as described herein.

FIG. 6A illustrates a simplified schematic and cross-sectional representation of an embodiment of a design hereof which provides for scaling up of devices, systems, and methods hereof by, for example, vertical or adjacent stacking of the channels or gas exchange units. A gas exchange unit, as used herein, is a combination of a gas channel(s) and adjacent liquid channel(s) between which gas transport can occur via one or more intermediate gas-permeable membranes. In the embodiment illustrated in FIG. 6A, the single channel concept described in connection with the above studies is expanded to include multiple gas channels and liquid channels. The gas channels are in fluid connection with one or more gas inlets which is/are in fluid connection with a source of sweep gas. The gas channels are also in fluid connection with one or more gas outlets. In the illustrated embodiment of FIG. 6A, each gas channel 108 serves to supply gas exchange to two liquid channels 112, resulting in four liquid channels 112 in total. Holes or passages in the rigid substrate/glass, represented by dashed lines in FIG. 6A, allow a single liquid inlet 112A to branch and thereby supply all liquid channels 112. Liquid inlet 112A and the vertically oriented inlet channel thereof may thus be considered to operate as an inlet manifold within the composite housing 102 of the device 100, while the liquid outlet 112B and the vertically oriented channel thereof operates as an outlet manifold within composite housing of the device 102. Body 114a or PDMS layer 1 is identical to the casting shown in FIG. 1A. Body 114b or PDMS layer 2 includes a casting of a liquid channel 112 on both the upper and lower sides thereof. Body 114c or PDMS layer 3 is similar to body 114a or layer 1, including a single liquid channel 112 formed therein. The pattern of layer 2/layer 3 can be repeated indefinitely to scale up the design as much as needed depending, for example, on limitations such as required pressure.

In FIG. 6A, an embodiment of a gas exchange system 50 hereof includes a gas exchange device 100 including a plurality of gas exchange units 105 in a stacked (vertically stacked in the illustrated orientation) arrangement. Each gas exchange unit 105 includes at least one gas channel 108 and at least one liquid channel 112, separated from an adjacent gas channel 112 by a gas-permeable membrane 120 through which gas may transport between the gas channel(s) and adjacent liquid channel(s). In the illustrated embodiment of FIG. 6A, each gas exchange unit 105 include a gas channel 108 and two liquid channels 112 adjacent thereto which are independently separated from gas channel 108 by a gas-permeable membrane 120, whereby gas transfer can occur between liquid channels 112 and gas channel 108. In other embodiments, a gas exchange unit hereof could include a single liquid channel and a single gas channel separated by a gas-permeable membrane or a single liquid channel and two adjacent gas channels separated from the liquid channel by independent gas-permeable membranes.

Each gas exchange unit 105 includes a substrate system 130 via which vibration/acoustic waves are transmitted to the one or more gas-permeable membranes 120 of gas exchange unit 105 via one or more oscillators 140. As described above, each gas permeable membrane 120 is connected to substrate system 130 and extends over an edge of a substrate section thereof to form a fluid connection with adjacent gas channel 108. Substrate system 130 can, for example, include a single, integral or monolithic section or sections of substrate material or separate, spaced sections of substrate. One or more oscillators can be provided in operative connection with each substrate section. Microchannel bodies 114a,b,c etc. or polymeric (body) layers (PDMS layers, for example), in which liquid channels 112 and gas channels 108 are formed, gas membranes 120 and substrate sections of substrate systems 130 are connected to stacked to form composite housing 102.

System 50 may, for example, include a control system 200 in operative connection with the device 100. Control system 200 may, for example, include a processor system 210. Processor system 210 may, for example, include one or more processors such as microprocessors as known in the art. A memory system 220 is in operative/communicative connection with processor system 210. Readable instructions (software components) are stored on the memory system and are executable by the processor system 210. A user interface system 230 is also provided in operative/communicative connection with processor system 210. Control system 200 may further include an input/output system 240, a communication system 250, and a sensor system 260 in operative connection with processor system 210 and a power system 270 (for example, a battery array in the case of an ambulatory device) to power components of control system 200. Sensor system 260 may, for example, include one or more sensors to measure data such as gas exchange rate, flow rate, temperature, sensor, etc. Closed-loop, feedback control of system 200 (including, for example, control of oscillation/vibration actuator(s) 130, and control of flow through device) may, for example, be achieved using data from one or more sensors of sensor system 260.

FIG. 6B illustrates an exploded perspective view of another embodiment of a gas exchange device 310 hereof including multiple, stacked gas exchange units 320 for gas exchange. In the illustrated embodiment, liquid channels 320 are formed in channel/microchannel bodies 314a, 314b, 314c which are, for example, formed from PDMS. In the illustrated embodiment, each liquid channel 320 includes a plurality of generally parallel subchannels. Body 314b includes an upper and a lower liquid channel 320 formed therein. Only upper liquid channel 320 of body 314b is illustrated in FIG. 6B. Passages through body 314b (not shown) at each lateral end thereof connect the two liquid channels 220.

Gas channels 330 are formed in a substrates system which, in the illustrated embodiment, includes substrate members 316a and 316b. As described above, substrate members 316a and 316b are formed from a material such as glass through which vibration/acoustic waves are efficiently transmitted. In the illustrated embodiment, each of substrate members 316a and 316b includes an upper and a lower (in the illustrated orientation) gas channel 230 formed therein. Further, each of substrate members 316a and 316b includes two actuators 340 (for example, piezo actuators) attached thereto at each end of upper gas channel 330. During actuation of both actuators 340, the primary direction of propagation of waves through membrane 350 would be in line with the direction of the gas flow (across the width of liquid channel 320), with wave components travelling potentially in both directions. In the illustrated embodiment, each gas channel 330 includes a plurality of spaced supports 332 extending therein (around which gas can pass). Supports 332 provide mechanical support from gas-permeable membranes 350 positioned between adjacent liquid channels 320 and gas channels 330. Each gas-permeable membrane 350 is pinned/attached to spaced sections of the associated substrate member 316a or 316b on each side of (and defining) gas channels 330 formed in substrate member 316a or 316b. Passages 318 are formed through substrate members 316a and 316b for flow of liquid therethrough to provide fluid connection between liquid channels 320 formed in channel bodies 314a, 314b and 314c and liquid inlet 320A and liquid outlet 320B. The flow of liquid through device 310 is represented by solid arrows L, while the flow of gas through device 310 is represented by dashed arrow G. Multiple devices 310 may, for example, be provided in a gas exchange system hereof (for example, a blood oxygenator). Each of liquid inlets 220A of such multiple devices 310 may, for example, be in fluid connection with a common, system liquid inlet/liquid inlet manifold. Each of liquid outlets 220B of such devices 310 may, for example, be in fluid connection with a common, system liquid outlet/liquid outlet manifold. Likewise, each of gas inlets and gas outlets of device 3210 may, for example, be in fluid connection with a common, system gas inlet/inlet manifold and a common, system outlet/outlet manifold, respectively.

In device 310, each of gas exchange units 312 includes one liquid channel and one adjacent gas channel 32—separated by a gas-permeable membrane 350 through which gas can transfer between an adjacent liquid channel 320 and gas channel 330. The stacked channel bodies 214a, 214b, and 214c, gas-permeable membranes 350, and substrate members 216a and 216b from a composite housing through which liquid and gas travel as illustrated.

A further series of experiments were performed to determine the effects of the acoustic streaming by oscillating the membrane on the hemocompatibility of a microchannel flow. First, surface fouling by platelet and protein deposition was investigated. Such deposition on the membrane may degrade gas transfer performance by physically blocking the porous avenues through which gas can travel. Some previous works have demonstrated that acoustic oscillation may improve performance in that regard.

In a number of studies, microchannel devices were fabricated as described above with a width of 1.8 mm and a height of either 250 μm or 600 μm, and with an orientation such that the oscillation travels in the direction perpendicular to the liquid flow, similar to the channels used in the experiments underlying the data in FIGS. 5A and 5B. The devices were placed on a hot plate set to 60° C. and a syringe pump was used to flow fresh ovine blood (citrated) through the channels at 0.1 mL/min for a total of 1.5 hr. The blood was citrated to ensure no coagulation would occur during these experiments and the effects of platelet deposition would be isolated and easier to observe. In each run, two channels were run side-by-side: one with the actuator on to generate acoustic streaming, and the other with no acoustic actuation.

In each side-by-side comparison (n=5 for each actuation status), surface fouling on the membrane by platelet deposition was significantly reduced for the actuated channel. Scanning electron microscope images were obtained at a few different areas of the surface for each sample. Images qualitatively showed reduced surface coverage of platelet deposition. For some of those images, an analysis was performed in software Image J to quantify the surface coverage, showing an average of around 22% coverage in the non-actuated channels vs 5% coverage in the actuated channels. The reduction is expected to be a result of the physical motion of the surface and vortex flows shaking loose platelets before they have an opportunity to bind to the surface.

Further blood flow experiments were performed with the types of microchannels described above to determine the effects of both the acoustic streaming and channel height on coagulation in the channel. Because the gas transfer device concept intends to use a taller microchannel with added actuation, it is beneficial to show an improvement in coagulation performance separately for each of those variables. Coagulation in a microchannel can take the form of smaller individual clumps of blood cells which do not grow very much over time (which will be referred to as “minor coagulation”), or it can take the form of complete blockage of the channel cross-section, leading to large increases in inlet pressure and catastrophic device failure (which will be referred to as “blockage”).

Because of the highly variable nature of the condition of the ovine blood used in this experimental setting, the parameters used for these experiments were also varied. In general, each trial was set up in an attempt to observe channel blockage beginning at around 20-40 min after the start of flow, to varying degrees of success. Either Heparin or citrate was used as an anticoagulant, where a minimum amount is necessary to enable the blood draw from the donor animal, but too strong an anticoagulant condition prevents us from observing coagulation in the channel itself. Raised temperature is usually necessary to ensure coagulation in the channel which was achieved either by placing samples directly on a hot plate between 60-90° C. or in a water bath with water temperature close to body temperature (37° C.). In certain tests, inlet pressure was measured by a manometer connected to the inlet before the syringe needle). Blood flow was input by syringe pump at 0.1 mL/min for up to 1 hr or until channel blockage.

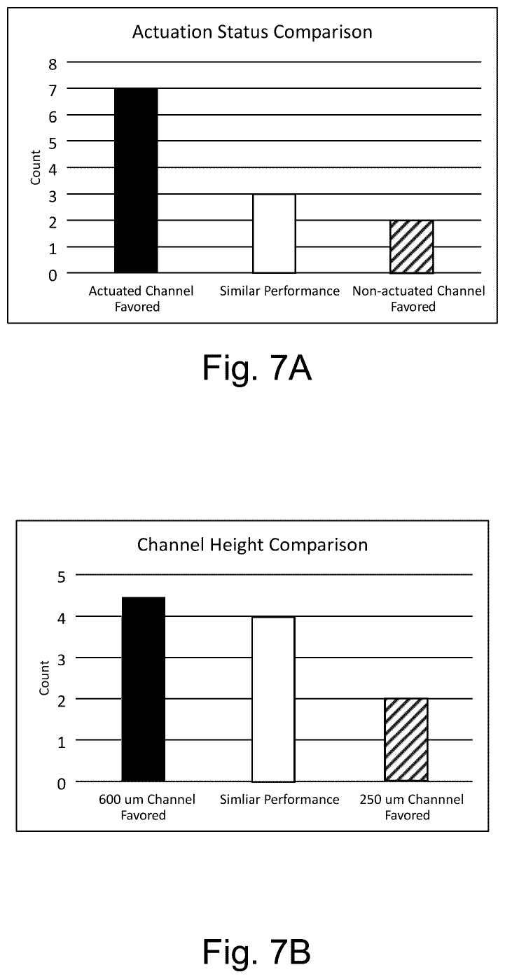

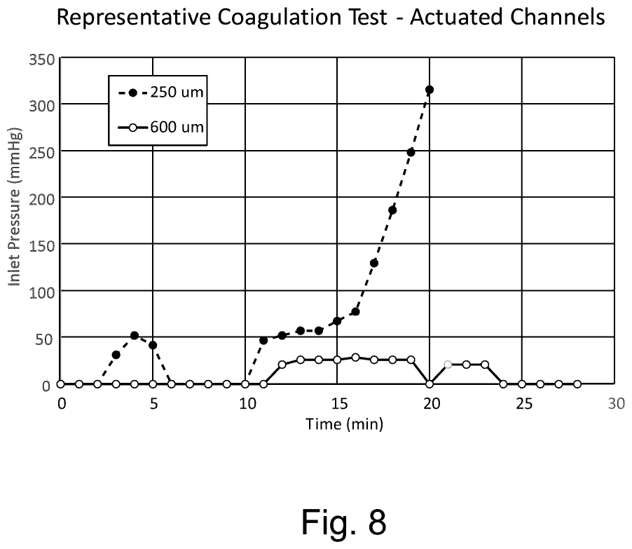

Using 2 channel heights (600 μm vs 250 μm) and 2 actuation statuses (on vs off) leads to 4 possible configurations. Since only two samples could be run simultaneously and because of the highly variable nature of the blood and resulting coagulation as described earlier, only samples run side-by-side were compared, and many individual comparisons were tabulated and summarized simply as a count of runs were one parameter performed better, worse, or about the same. The bar chart of FIG. 7A presents the compendium of those side-by-side comparisons and shows a clear reduction in coagulation (be it by minor coagulation or full blockage) for an actuated channel vs a non-actuated channel. Also, a lesser, yet still significant reduction in coagulation is seen in the taller 600 μm channel vs the shorter 250 μm channel as illustrated in FIG. 7B. Further, a representative plot is provided in FIG. 8 for a test procedure in which inlet pressure was measured, which shows improved performance in a taller 600 μm channel compared to a shorter 250 μm channel, both with actuation. For the shorter channel, the initiation of blockage (as measured by the first increase read on the manometer) occurs in less than half the time than for the taller channel. Further, the shorter channel eventually undergoes irreversible blockage as shown by the eventual exponential increase in pressure. Some pressure increase was measurable in the taller channel. However, blood was able to flow through until the sample volume of blood was exhausted over the 28 minutes of the experiment.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A device, comprising:
a housing:
a gas inlet in connection with the housing;
a gas outlet in connection with the housing;
a liquid inlet in connection with the housing;
a liquid outlet in connection with the housing,
one or more gas exchange units within the housing, each gas exchange unit comprising
a gas channel within the housing and in fluid connection with the gas inlet and with the gas outlet; and
either a first liquid channel in fluid connection with the liquid inlet and with the liquid outlet or the first liquid channel and a second liquid channel in fluid connection with the liquid inlet and with the liquid outlet, the first liquid channel being positioned adjacent to the gas channel on a first side thereof, the second liquid channel, when present, being positioned adjacent to the gas channel on a second side thereof, opposite the first side, the first liquid channel being separated from the gas channel via a first gas-permeable membrane so that gas may transport between the first liquid channel and the gas channel via the first gas-permeable membrane, the second liquid channel, when present, being separated from the gas channel via a second gas-permeable membrane so that gas may transport between the second liquid channel and the gas channel via the second gas-permeable membrane, the first gas-permeable membrane being connected to a rigid substrate system so that the first gas-permeable membrane extends beyond a first edge of the rigid substrate system, the second gas-permeable membrane, when present, being connected to the rigid substrate system so that the second gas-permeable membrane extends beyond a second edge of the rigid substrate system; and
an oscillator system comprising one or more oscillators in operative connection with the rigid substrate system, the oscillator system being configured to induce oscillation in the rigid substrate system and thereby in the first gas-permeable membrane and the second gas-permeable membrane, when present.

2. The device of claim 1 wherein each of the first liquid channel and the second liquid channel, when present, has a height no greater than 2 mm.

3. The device of claim 2 wherein each of the first liquid channel and the second liquid channel, when present, has a height of at least 50 μm.

4. The device of claim 2 wherein each of the first liquid channel and the second liquid channel, when present, has a height of at least 200 μm.

5. The device of claim 2 wherein each of the first liquid channel and the second liquid channel, when present, has a height in the range of 200 μm to 1 mm.

6. The device of claim 2 wherein a frequency of oscillation of each of the one or more oscillators is controlled to be in the range of 1 kHz to 20 kHz.

7. The device of claim 2 wherein a wavelength of oscillation induced in the first gas-permeable membrane is greater than any dimension of the first liquid channel and greater than any dimension of the second liquid channel, when present.

8. The device of claim 6 wherein a wavelength of oscillation is at least 10 times the dimension of the first liquid channel in the direction in which waves oscillate through the first gas-permeable membrane, and at least 10 times the dimension of the second liquid channel, when present, in the direction in which waves oscillate through the second gas permeable membrane.

9. The device of claim 6 wherein a wavelength of oscillation is at least 100 times the dimension of the first liquid channel in the direction in which waves oscillate through the first gas-permeable membrane, and at least 100 times the dimension of the second liquid channel, when present, in the direction in which waves oscillate through the second gas permeable membrane.

10. The device of claim 2 wherein a direction of bulk flow of gas through the gas channel is oriented generally perpendicular to bulk flow of liquid through the first liquid channel and through the second liquid channel, when present.

11. The device of claim 2 wherein the first gas-permeable membrane extends from a first edge of a first section of the rigid substrate system, and the second gas-permeable membrane extends from a second edge of the first section of the rigid substrate system, which is opposite the first edge.

12. The device of claim 2 wherein a space between a first section of the rigid substrate system and a second section of the rigid substrate system forms the gas channel, the first gas-permeable membrane being connected to the first section and to the second section of the rigid substrate system to span the gas channel.

13. The device of claim 2 comprising a plurality of the gas exchange units.

14. The device of claim 13 wherein the plurality of gas exchange units are positioned in a stacked arrangement.

15. The device of claim 13 wherein each of the plurality of gas exchange units has a separate oscillator in operative connection with the rigid substrate system thereof.

16. The device of claim 13 wherein the oscillator is in operative connection with rigid substrate of more than one of the plurality of gas exchange units.

17. A method of effecting gas exchange between a liquid and a sweep gas, comprising:
providing a device comprising:
a housing;
a gas inlet in connection with the housing;
a gas outlet in connection with the housing;
a liquid inlet in connection with the housing;
a liquid outlet in connection with the housing,
one or more gas exchange units within the housing, each gas exchange unit comprising
a gas channel within the housing and in fluid connection with the gas inlet and with the gas outlet; and
either a first liquid channel in fluid connection with the liquid inlet and with the liquid outlet or the first liquid channel and a second liquid channel in fluid connection with the liquid inlet and with the liquid outlet, the first liquid channel being positioned adjacent to the gas channel on a first side thereof, the second liquid channel, when present, being positioned adjacent to the gas channel on a second side thereof, opposite the first side, the first liquid channel being separated from the gas channel via a first gas-permeable membrane so that gas may transport between the first liquid channel and the gas channel via the first gas-permeable membrane, the second liquid channel, when present, being separated from the gas channel via a second gas-permeable membrane so that gas may transport between the second liquid channel and the gas channel via the second gas-permeable membrane, the first gas-permeable membrane being connected to a rigid substrate system so that the first gas-permeable membrane extends beyond a first edge of the rigid substrate system, the second gas-permeable membrane, when present, being connected to the rigid substrate system so that the second gas-permeable membrane extends beyond a second edge of the rigid substrate system; and
an oscillator system comprising one or more oscillators in operative connection with the rigid substrate system, the oscillator system being configured to induce oscillation in the rigid substrate system and thereby in the first gas-permeable membrane and the second gas-permeable membrane, when present;
passing liquid through the first liquid channel and the second liquid channel, when present, via the liquid inlet and the liquid outlet; and
passing gas through the gas channel via the gas inlet and the gas outlet.

18. The method of claim 17 wherein each of the first liquid channel and the second liquid channel, when present, has a height no greater than 2 mm.

19. The method of claim 18 wherein each of the first liquid channel and the second liquid channel, when present, has a height of at least 50 μm.

20. The method of claim 18 wherein each of the first liquid channel and the second liquid channel, when present, has a height of at least 200 μm.

21. The method of claim 18 wherein each of the first liquid channel and the second liquid channel, when present, has a height in the range of 200 μm to 1 mm.

22. The method of claim 18 wherein a frequency of oscillation of each of the one or more oscillators is controlled to be in the range of 1 kHz to 20 kHz.

23. The method of claim 18 wherein a wavelength of oscillation induced in the first gas-permeable membrane is greater than any dimension of the first liquid channel and greater than any dimension of the second liquid channel, when present.

24. The method of claim 18 wherein the device comprises a plurality of the gas exchange units.

25. The method of claim 24 wherein the plurality of gas exchange units are positioned in a stacked arrangement.

26. The method of claim 18 wherein the liquid is blood and the gas comprises oxygen.

27. A system comprising a plurality of devices, each of the plurality of devices, comprising:
a housing;
a gas inlet in connection with the housing;
a gas outlet in connection with the housing;
a liquid inlet in connection with the housing;
a liquid outlet in connection with the housing,
one or more gas exchange units within the housing, each gas exchange unit comprising
a gas channel within the housing and in fluid connection with the gas inlet and with the gas outlet; and
either a first liquid channel in fluid connection with the liquid inlet and with the liquid outlet or the first liquid channel and a second liquid channel in fluid connection with the liquid inlet and with the liquid outlet, the first liquid channel being positioned adjacent to the gas channel on a first side thereof, the second liquid channel, when present, being positioned adjacent to the gas channel on a second side thereof, opposite the first side, the first liquid channel being separated from the gas channel via a first gas-permeable membrane so that gas may transport between the first liquid channel and the gas channel via the first gas-permeable membrane, the second liquid channel, when present, being separated from the gas channel via a second gas-permeable membrane so that gas may transport between the second liquid channel and the gas channel via the second gas-permeable membrane, the first gas-permeable membrane being connected to a rigid substrate system so that the first gas-permeable membrane extends beyond a first edge of the rigid substrate system, the second gas-permeable membrane, when present, being connected to the rigid substrate system so that the second gas-permeable membrane extends beyond a second edge of the rigid substrate system; and
an oscillator system comprising one or more oscillators in operative connection with the rigid substrate system, the oscillator system being configured to induce oscillation in the rigid substrate system and thereby in the first gas-permeable membrane and the second gas-permeable membrane, when present.
